(19) United States
(12) Patent Application Publication
(10) Pub. No.: US 2010/0152499 A1
(43) Pub. Date: Jun. 17, 2010

(54) CONVERSION OF A MULTIHYDROXYLATED-ALIPHATIC HYDROCARBON OR ESTER THEREOF TO A CHLOROHYDRIN

(76) Inventors: John R. Briggs, Midland, MI (US); Bruce D. Hook, Lake Jackson, TX (US); William J. Kruper, JR., Sanford, MI (US); Anil Mehta, Lake Jackson, TX (US); Robert M. Alvarado, Lake Jackson, TX (US); Sascha Noormann, Gruenendeich (DE); Perry S. Basile, Lake Jackson, TX (US)

Correspondence Address: The Dow Chemical Company, P.O. BOX 1967, Midland, MI, 48641, US

(21) Appl. No.: 12/595,396

(22) PCT Filed: Apr. 11, 2008
(86) PCT No.: PCT/US08/59977

§ 371 Date: Jan. 20, 2010

Related U.S. Application Data
(60) Provisional application No. 60/923,055, filed on Apr. 12, 2007.

(51) Int. Cl. C07C 31/36; B01J 19/00

(52) U.S. Cl. 568/844; 422/129

(57) ABSTRACT

The present invention relates to a process for converting at least one multihydroxylated-aliphatic hydrocarbon and/or an ester thereof to at least one chlorohydrin and/or an ester thereof, comprising at least one reaction step in which the multihydroxylated-aliphatic hydrocarbon and/or ester thereof is contacted with hydrogen chloride under reaction conditions to produce the chlorohydrin and/or ester thereof, followed by at least one downstream processing step in which the effluents of the reaction step are processed, wherein the downstream processing step is performed in such conditions that the effluents containing the chlorohydrin and/or ester thereof are kept at a temperature of less than 12O° C. The invention allows to minimize the liberation of hydrogen chloride from the products of the hydrochlorination reaction, hence reducing the corrosion of the downstream equipment and reducing M the need to use costly corrosion resistant materials.

### BACKGROUND OF THE INVENTION

The present invention relates to a process for converting a multihydroxylated-aliphatic hydrocarbon or an ester thereof to a chlorohydrin. Chlorohydrins, in turn, are useful in preparing epoxides such as epichlorohydrins.

Epichlorohydrin is a widely used precursor to epoxy resins. Epichlorohydrin is a monomer which is commonly used for the alkylation of para-bisphenol A; the resultant diepoxide, either as a free monomer or oligomeric diepoxide, may be advanced to high molecular weight resins which are used for example in electrical laminates, can coatings, automotive topcoats and clearcoats.

A known process for the manufacture of epichlorohydrin involves hypochlorination of allyl chloride to form dichlorohydrin. Ring closure of the dichlorohydrin mixture with caustic affords epichlorohydrin which is distilled to high purity (>99.6%). This chlorohydrin process requires two equivalents of chlorine and one equivalent of caustic per molecule of epichlorohydrin.

In another known process for producing epichlorohydrin the first step involves installing oxygen in the allylic position of propylene, via a palladium catalyzed reaction of molecular oxygen in acetic acid. The resulting allyl acetate is then hydrolyzed, chlorinated and the incipient dichlorohydrin is ring closed with caustic to epichlorohydrin. This process avoids the production of allyl chloride and therefore uses less chlorine (only one equivalent).

Both known processes for the manufacture of epichlorohydrin described above require the sacrificial use of chlorine, and complications associated with the industrial use and generation of hypochlorous acid (HOCl) can be magnified at industrial scale and these processes are known to produce substantial amounts of chlorinated by-products. In particular, it is well known that the hypochlorination of allyl chloride produces 1,2,3-trichloropropane and other undesirable chlorinated ethers and oligomers (RCls). RCl issues are managed as an increased cost to manufacture. As new capital is added to accommodate greater global production, a substantial investment in downstream processing must be added to accommodate and remediate these unwanted by-products. These same problems are analogous in the HOCl routes to propylene and ethylene chlorohydrin, and thus, these routes are less practiced.

An alternative process, which avoids the generation of HOCl, for example as described in patent application WO 2002/092586 and U.S. Pat. No. 6,288,248 involves the direct epoxidation of allyl chloride using titanium silicalite catalysis with hydrogen peroxide. Despite the advantage of reducing the generation of HOCl, allyl chloride is still an intermediate. The disadvantage of using allyl chloride is two-fold: (1) The free radical chlorination of propylene to allyl chloride is not very selective and a sizable fraction (>15 mole %) of 1,2-dichloropropane is produced. (2) Propylene is a hydrocarbon feedstock and long-term, global forecast of propylene price continues to escalate. A new, economically viable process for the production of epichlorohydrin which avoids the complications of controlled, chlorine-based oxidation chemistry and RCl generation is desirable. There is a need in the industry for a process for the generation of epichlorohydrin which involves a non-hydrocarbon, renewable feedstock.

Glycerin is considered to be a low-cost, renewable feedstock which is a co-product of the biodiesel process for making fuel additives. It is known that other renewable feedstocks such as fructose, glucose and sorbitol can be hydrogenolized to produce mixtures of vicinal diols and triols, such as glycerin, ethylene glycol, 1,2-propylene glycol, 1,3-propylene glycol and the like.

With abundant and low cost glycerin or mixed glycols, an economically attractive process for glycerin or mixed glycol hydrochlorination would be desirable. It would be advantageous if such a process were highly chemoselective to the formation of vicinal chlorohydrins, without production of RCls.

A process is known for the conversion of glycerol (also referred to herein as “glycerin”) to mixtures of dichloropropanols (also referred to herein as “dichlorohydrins”), compounds I and II, as shown in Scheme 1 below. The reaction is carried out in the presence of anhydrous HCl and an acetic acid (HOAc) catalyst with water removal. Both compounds I and II can then be converted to epichlorohydrin via treatment with caustic.

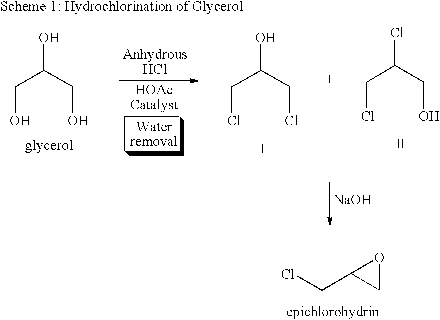

Various processes using the above chemistry in Scheme 1 have been reported in the prior art. For example, epichlorohydrin can be prepared by reacting a dichloropropanol such as 2,3-dichloropropan-1-ol or 1,3-dichloropropan-2-ol with base. Dichloropropanol, in turn, can be prepared at atmospheric pressure from glycerol, anhydrous hydrochloric acid, and an acid catalyst. A large excess of hydrogen chloride (HCl) gas is recommended to promote the azeotropic removal of water that is formed during the course of the reaction.

For example, Gibson, G. P., Chemistry and Industry 1931, 20, 949-975; and Conant et al., Organic Synthesis CV 1, 292-294, and Organic Synthesis CV 1, 295-297; have reported distilled yields of dichlorohydrins in excess of 70% for dichlorohydrins, compounds I and II in Scheme 1 above, by purging a large excess of anhydrous HCl (up to 7 equivalents) through a stirred solution of glycerol and an organic acid catalyst. The processes described in the above references require the use of atmospheric pressures of HCl which is used as an azeotroping agent to remove the accumulated water. Other azeotropes are known. For example, U.S. Pat. No. 2,144,612 describes using n-butyl ether along with excess hydrogen chloride (HCl) gas to promote the reactive distillation and removal of water.

Indeed, all of the prior art teaches the vaporization of azeotropes with water to provide high conversion and a process need for sub-atmospheric or atmospheric pressure conditions to accomplish water removal. U.S. Pat. No. 2,144,612 argues the advantageous use of an added azeotroping agent (for example, n-butyl ether) to promote the reactive azeotropic distillation and elimination of water, again using excess HCl at atmospheric conditions. A similar approach using vacuum removal of water is taught in German Patent No. 1075103.

German Patent No. 197308 teaches a process for preparing a chlorohydrin by the catalytic hydrochlorination of glycerine by means of anhydrous hydrogen chloride. This reference teaches a batch process with separation of water at atmospheric conditions. German Patent No. 197308 does not teach carrying out the hydrochlorination reaction process at elevated pressures.

All known prior art for the production of chlorohydrin reports hydrochlorination processes where water is removed as a co-product from the process. In particular, WO 2005/021476 teaches a series of hydrochlorination reactions in which the water of reaction is removed in an atmospheric or sub-atmospheric process by reactive distillation. Similar art is taught in WO2005/054167 with the additional teaching that the reaction carried out under higher total pressures (HCl partial pressure not specified) may improve the rate of reaction. However, nothing in WO2005/054167 discloses the use of HCl partial pressure and its effect in its process. WO2005/054167 also exemplifies the need to remove water to effect high conversion and selectivity under atmospheric or subatmospheric pressures. Neither WO 2005/021476 nor WO2005/054167 teaches any advantage of leaving water in their processes, or that removing the water effects the formation of unwanted chloroethers and RCl's.

The use of extremely large excess amounts of hydrogen chloride (HCl) gas is economically problematic and the inherent contamination with water of the unreacted hydrogen chloride results in an aqueous hydrogen chloride stream that is not easily recyclable. Furthermore, reaction times of 24 to 48 hours are required to achieve a far from complete conversion of glycerin; however, the products often include significant amounts of the undesired overchlorinated trichloropropane and chlorinated ethers. Other processes are also known that use reagents that convert alcohols to chlorides but that scavenge water in situ. For example, thionyl chloride can be used to convert glycerin to a chlorohydrin, as described in Cane, Mauclere C. R. Hebd. Seances Acad. Sci. 1930, 192 and may be selective, but produces stoichiometric amounts of SO₂. The cost and expense of this reagent is not acceptable for the industrial production of epichlorohydrin or any other chlorohydrin derived from a multihydroxylated-aliphatic hydrocarbon Likewise, other hydrochlorination reagents which are mild and effective are considered expensive and exotic for this transformation, as described in Gomez, et al. Tetrahedron Letters 2000, 41, 6049-6052. Other low temperature processes convert the alcohol to a better leaving group (for example, mesylate) and provide a soluble form of chloride via an ionic liquid used in molar excess, as described in Leadbeater, et al. Tetrahedron 2003, 59, 2253-58. Again, the need for anhydrous conditions, stoichiometric reagents and an expensive form of chloride prevents industrial consideration of the above process. Furthermore, these reagents can cause exhaustive chlorination of a multihydroxylated-aliphatic hydrocarbon, leading again to undesirable RCl by-products, as taught in Viswanathan, et al. Current Science, 1978, 21, 802-803.

To summarize, there are at least five major disadvantages to all of the above known approaches for preparing a chlorohydrin from glycerin or any other vicinal-diol, triol or multihydroxylated-aliphatic hydrocarbon: (1) Atmospheric pressure processes for the hydrochlorination of glycerin or any diol require a large excess of HCl, oftentimes 7-10 fold molar excess. In an atmospheric pressure process the excess anhydrous HCl is then contaminated with water. (2) Variants of the above known processes are very slow, batch type reactions, which often take between 24-48 hours at temperatures in excess of 100° C. and do not exceed 80-90% conversion to desired chlorohydrin product(s). (3) Exotic hydrochlorination reagents may drive the reaction by scavenging water, but oftentimes produce a by-product inconsistent with the economic production of a commodity. (4) All of the above approaches produce higher levels of unwanted RCls, as defined above for glycerin hydrochlorination. (5) When the reaction is run at elevated pressure to control vaporization of the reactor contents, low partial pressures of HCl result in low conversions or retarded reaction rates.

The prior art concludes that water removal is required to promote complete conversion of glycerin to dichlorohydrins. To accommodate this water removal requirement, the prior art reactions are conducted under azeotropic or reactive distillation or extraction conditions which requires a co-solvent or chaser and considerable capital addition to the process. All prior art has concluded that there is an equilibrium limitation to this conversion due to the presence of water in the reaction mixture.

It is desired in the industry to provide a hydrochlorination process for the production of high purity chlorohydrins from multihydroxylated-aliphatic hydrocarbons which overcome all of the inadequacies of the prior art. It would, therefore, be an advance in the art of chlorohydrin chemistry to discover a simple and cost-effective method of transforming diols and triols to chlorohydrins.

It is also known that the process of the hydrochlorination of multihydroxylated aliphatic hydrocarbon forms a corrosive medium. For example, patent application WO2006/020234 discloses that the equipment useful for the hydrochlorination reaction may be any well-known equipment in the art and should be capable of containing the reaction mixture at the conditions of the hydrochlorination. Suitable equipment may be fabricated of materials which are resistant to corrosion by the process components, and may include for example, metals, such as tantalum, suitable metallic alloys such as Hastalloy C™, or glass-lined equipment.

It is also known that processes that employ hydrogen chloride, particularly in the presence of water and/or alcohols form a corrosive medium, and that such processes require the use of corrosion resistant materials to adequately contain the reaction mixtures. For example U.S. Pat. No. 4,701,226 discloses that tantalum and glass-lined equipment is resistant to acidic environments (column 1, line 26). This same document summarizes an earlier reference by Ruf and Tsuei (J. of Applied Physics, Vol. 54, No. 10, page 5703, (1983)) which report that although amorphous chromium is rapidly corroded by 12 N hydrochloric acid, the addition of boron to the chromium gives a very corrosive-resistant alloy. Also reported (column 2, line 27) is that alloys that may appear corrosion resistant to hydrochloric acid at room temperature can be unsuitable at higher temperatures.

Kirk Othmer Encyclopedia of Chemical Technology, 3^{rd} Edition, John Wiley and Sons, publishers, 1980, incorporated herein by reference, reports (vol 12, page 1003) that most metals react with aqueous hydrochloric acid and that the rate of corrosion depends on a variety of factors, including the temperature, the concentration of the acid, the presence of inhibitors and the nature of the metal surface. On page 1003, tantalum and zirconium are reported to be resistant to HCl, but the latter fails in the presence of ferric or cupric ions. Nickel alloys, particularly nickel-molybdenum alloys, including Hastelloy™ (trademark of High Performance Alloys, Inc.) are recommended for hot service (page 1003). Tungsten and molybdenum are reported to exhibit good room temperature resistance to corrosion, but fail at 100° C. On page 831, a table reports the resistance of a variety of metals and graphite to hydrochloric acid. Also reported (ibid, page 1003) is that common plastics and elastomers show excellent resistance to hydrochloric acid within the temperature limits of the materials. Polymers that are reported to exhibit some resistance to hydrochloric acid include natural rubber, neoprene, nitrile, butyl, chlorobutyl, hyperlon, ethylene-propylene-diene (EPDM), polypropylene, poly(vinyl chloride), Saran, acrylonitrile-butadiene-styrene (ABS) and fluorocarbon plastics. Fluorocarbon plastics are identified as having extremely high resistance to hydrochloric acid and a high temperature limit of operation. Carbon and graphite rendered impervious by impregnation with phenolic, epoxy or furan resins are identified as being suitable for hydrochloric acid service up to 170° C. The use of these carbon or graphite materials for use in heat exchanges and centrifugal pumps is disclosed.

Glass and ceramic lined equipment, and refractories of alumina, silica, zirconia and chrome-alumina are also described as suitable material for hydrochloric acid service.

Kirk Othmer Encyclopedia of Chemical Technology, 2^{nd} Edition, John Wiley and Sons publishers, 1966 volume 11 presents an extensive discussion of the corrosion resistance of a long list of metals and non-metals that can be used in hydrochloric acid and hydrogen chloride service (volume 11, page 323-327).

It is therefore well known in the art that hydrogen chloride and hydrochloric acid are corrosive to many metallic materials. Processes using hydrochloric acid or hydrogen chloride gas generally must employ equipment that is resistant to the corrosive medium that often exists in such chemical processes. The hydrochlorination of multihydroxylated-aliphatic hydrocarbons to chlorohydrins with hydrochloric acid or hydrogen chloride gas is an example of a process that forms a corrosive medium, as taught for example in patent applications WO 2005/054167 and in WO 2006/020234. These applications disclose the use of materials in the hydrochlorination reactor that are resistant to the hydrochlorinating agent, hydrogen chloride, which include glass-lined steel, tantalum, precious metals such as gold and polymers. WO 2005/054167 discloses (page 6, line 4) that “The process for producing a chlorinated organic compound according to the invention is generally carried out in a reactor made of or coated with materials that are resistant, under the reaction conditions, to the chlorinating agents, in particular hydrogen chloride.” Following this is a list of suitable materials.

WO 2006/020234 discloses (page 21, line 28) that “The equipment useful for the hydrochlorination reaction may be any-well known equipment in the art and should be capable of containing the reaction mixture at the condition of the hydrochlorination. Suitable equipment may be fabricated of materials which are resistant to corrosion by the process components, and may include, for example, metals, such as tantalum, suitable metallic alloys, such as Hastelloy ©, or glass-lined equipment. Suitable equipment may include, for example, single or multiple stirred tanks, tubes or pipes or combinations thereof.”

Furthermore, WO 2006/100317 discloses that in a process for the hydrochlorination of multihydroxylated-aliphatic hydrocarbons using hydrogen chloride, corrosion can occur in equipment downstream of the hydrochlorination process itself. The experimental data in WO 2006/100317 shows that some metals (example 1) are dissolved by 0.8 weight % of HCl in an aqueous mixture of some of the hydrochlorination reaction products. Example 2 shows that PTFE (poly(tetrafluoroethylene), graphite and enameled steel are not dissolved by the same mixture. The materials that are not affected by this medium are materials that have been previously disclosed as being resistant to hydrogen chloride in the prior art.

In particular, WO 2006/100317 teaches that steps of the hydrochlorination process beyond the hydrochlorination step are subject to corrosion and thus should preferably be performed in equipments made of, or covered with, corrosion resistant materials.

The use of corrosion resistant materials in service where exposure to corrosion will occur, for example where it is in contact with process streams known to contain hydrochloric acid or hydrogen chloride, it is desired to minimize dissolution of the equipment in the process stream, to minimize contamination of the process stream with the products of equipment corrosion, and to minimize maintenance and replacement costs.

On the other hand, the use of corrosion resistant materials in equipment which is not subject to corrosion is not desired, due to the increased cost of equipment made of such corrosion resistant materials. Additionally, such equipment, e.g. glass-lined reactors and pipes, are more fragile than equipment fabricated from conventional, non-resistant materials, and may suffer a greater failure rate due to physical events, e.g. movement, than conventional equipment.

It is desirable therefore to employ corrosion-resistant materials only where they are required due to contact with process streams that cause an unacceptable level of corrosion of the equipment. Where corrosion resistant material is not required due to the absence of contact with corrosion causing process streams, e.g. hydrochloric acid or hydrogen chloride, it is preferred to employ equipment fabricated from less expensive, conventional materials.

Finally, it is known in the art that hydrogen fluoride reacts with glass to generate silicon tetrafluoride (Kirk-Othmer, 3^{rd} Edition, Jon Wiley publishers, Volume 10, page 746), which leads dissolution of glass or glass-lined materials. In the hydrochlorination process, hydrogen fluoride can be formed from the reaction of fluoride ions with acids such as sulfuric acid or hydrochloric acid. Thus, it is desirable to avoid the formation of hydrogen fluoride at every stage of the hydrochlorination process.

### SUMMARY OF THE INVENTION

One aspect of the invention is the identification of conditions wherein the products of a process for the hydrochlorination of a multihydroxylated-aliphatic hydrocarbon are stable against the formation of acidic solutions.

A second aspect of the invention is the use of equipment fabricated from appropriate materials of construction employed to contain the products of the hydrochlorination of a multihydroxylated-aliphatic hydrocarbon depending upon the conditions at which the products are stored, or their thermal history.

A third aspect of the invention is the process of treating the product of the hydrochlorination of a multihydroxylated-aliphatic hydrocarbon which has formed an acidic medium because of its thermal history, to reduce its acidity and render it less corrosive to non-resistant materials of construction.

A fourth aspect of the invention is the control of the process contaminants such as fluorine to prevent the dissolution of equipment throughout the hydrochlorination process.

### DESCRIPTION OF THE INVENTION

We have surprisingly discovered that the products of the hydrochlorination of a multihydroxylated-aliphatic hydrocarbon become acidic upon heating. Although not wishing to be bound by theory, we believe that the hydrochlorination process products liberate hydrogen chloride upon heating. This liberated hydrogen chloride renders the hydrochlorination process products acidic and corrosive to non-resistant materials which are in contact with the material. The acidity and hence the corrosivity of the products are thus dependent on the temperature history of the product stream.

We have surprisingly discovered that downstream equipment need not be resistant to corrosion under the conditions of use, depending upon the conditions at which the products of the process are maintained. Under preferred conditions the stability of the hydrochlorination products is such that observed levels of corrosion are not detrimental to the hydrochlorination process or product, and that the increased cost of fabricating downstream process equipment of resistant materials does not justify their selection over materials that show less than complete resistance to the hydrochlorinating agent.

We have now determined conditions which lead to the liberation of hydrogen chloride from the multihydroxylated-aliphatic hydrocarbon, and conversely, conditions where liberation of hydrogen chloride is limited. Where liberation of hydrogen chloride is limited, the downstream equipment in contact with these process streams can be fabricated from materials of construction of less-resistant or non-resistant materials without detrimental effect on the process or product. In such downstream process equipment, while corrosion may still occur, its occurrence does not justify the installation of resistant materials of construction due to their increased cost, difficulty in fabrication and increased cost of maintenance.

The acidity of aqueous solutions is commonly measured by the pH scale. The pH of an aqueous solution is the negative base 10 logarithm of the hydrogen ion concentration. Thus by measuring the pH of aqueous hydrogen chloride, one can readily determine the concentration of HCl. For example, a 0.8 weight % solution of hydrogen chloride in water would give a pH of 0.66. An aqueous solution of hydrogen chloride exhibiting a pH of 1 contains 0.37 weight % hydrogen chloride.

In process streams where the concentration of hydrogen chloride, either as a gas or in solution is less than about 0.8% by weight, corresponding to an aqueous pH greater than 0.7 it may not be necessary to employ resistant materials. The hydrogen chloride may be present because it is intentionally added, because it is carried over from an earlier or later part of the process, or may be formed due to liberation from the product of hydrochlorination of the multihydroxylated-aliphatic hydrocarbon upon heating.

We have found that the liberation of hydrogen chloride occurs both in the presence and the absence of the hydrochlorination process carboxylic acid catalyst or its esters for the hydrochlorination at temperatures of 120° C. and above. Similarly, as the temperature is reduced below 120° C., the liberation of hydrogen chloride from the product of hydrochlorination of the multihydroxylated-aliphatic hydrocarbon is minimized

It is further known that water may exacerbate the corrosive effect of hydrogen chloride, and its reduction mitigates the corrosive effect. It may be advantageous to minimize the concentration of water in downstream equipment, since this may contribute to an increased rate of corrosion of non-resistant materials.

Furthermore, we have found that the acidity of the products of the hydrochlorination reaction should be reduced to below 0.8% by weight of hydrogen chloride, corresponding to an aqueous pH of greater than 0.66, or to a level where non-resistant materials of construction may be employed thereafter.

Finally, we have found that it is important to keep the fluoride concentration in the hydrochlorination process as low as possible to prevent the dissolution of equipment throughout the hydrochlorination process, particularly that which is protected by a glass lining or coating. In particular, the total fluoride concentration in the process should be limited to less than 50 ppm by weight.

### BRIEF DESCRIPTION OF THE DRAWINGS

FIG. 1 is a process flowchart illustrating one embodiment of the process of the present invention referred to herein as a once-through, no recycle process.

FIG. 2 is a process flowchart illustrating another embodiment of the process of the present invention referred to herein as a catalyst and intermediate recycle process.

FIG. 3 is a process flowchart illustrating another embodiment of the process of the present invention referred to herein as a catalyst and intermediate recycle process with transesterification.

### DETAILED DESCRIPTION OF THE INVENTION

In one broad aspect, the present invention relates to a process for converting at least one multihydroxylated-aliphatic hydrocarbon and/or an ester thereof to at least one chlorohydrin and/or an ester thereof, comprising at least one reaction step in which the multihydroxylated-aliphatic hydrocarbon and/or ester thereof is contacted with hydrogen chloride under reaction conditions to produce the chlorohydrin and/or ester thereof, followed by at least one downstream processing step in which the effluents of the reaction step are processed, wherein the downstream processing step is performed in such conditions that the effluents containing the chlorohydrin and/or ester thereof are kept at a temperature of less than 120° C.

In a second aspect, the present invention relates to a process for reducing corrosion in the equipment located downstream of a hydrochlorination reaction zone in which at least one multihydroxylated-aliphatic hydrocarbon and/or an ester thereof is converted into at least one chlorohydrin and/or an ester thereof, wherein the effluents of the reaction zone containing the chlorohydrin and/or ester thereof are kept at a temperature of less than 120° C.

In a third aspect, the present invention relates to an installation for converting at least one multihydroxylated-aliphatic hydrocarbon and/or an ester thereof to at least one chlorohydrin and/or an ester thereof, comprising at least one reaction unit in which the multihydroxylated-aliphatic hydrocarbon and/or ester thereof is contacted with hydrogen chloride under reaction conditions to produce the chlorohydrin and/or ester thereof, said reaction unit being connected to at least one downstream processing unit in which the effluents of the reaction unit are processed and/or stored, wherein the equipment used in said downstream processing unit is made of or covered with corrosion resistant material in the only areas where such equipment is in contact with an effluent whose total hydrogen chloride concentration is greater than 0.8% by weight, relative to the total weight of said effluent.

According to an advantageous embodiment of the invention, the effluents of the hydrochlorination reaction step containing the chlorohydrin or ester thereof are kept at a temperature of less than 100° C., more preferably of less than 90° C.

By keeping as low as possible the temperature at which the chlorohydrin or ester thereof from the reaction step are further processed, the invention allows to minimize the liberation of hydrogen chloride from such products, hence reducing the corrosion of the downstream equipment and lengthening the service life of such equipment. Furthermore, as corrosion of the downstream equipment is reduced, the invention reduces the need to use costly corrosion resistant material.

According to a first embodiment of the invention, the downstream processing equipment used in said downstream processing step is made of or covered with corrosion resistant material in the only areas where such downstream processing equipment is in contact with an effluent whose total hydrogen chloride concentration is greater than 0.8% by weight, relative to the total weight of said effluent.

According to this first embodiment, in the areas where the downstream processing equipment is in contact with an effluent whose total hydrogen chloride concentration is under 0.8% by weight, said downstream processing equipment is not made of or covered with corrosion resistant material. Hence, the downstream processing equipment is made of or covered with corrosion resistant material only where it is in contact with an effluent having a total hydrogen chloride concentration greater than 0.8% by weight, relative to the total weight of said effluent.

As used therein, the term “effluent of the reaction step” refers to any compound or mixture of compounds coming directly or indirectly from the reaction step.

The effluents can contain for example and non limitatively at least one compound chosen from chlorohydrin, esters of chlorohydrin, water, catalyst, remaining multihydroxylated-aliphatic hydrocarbon and/or ester thereof, remaining hydrogen chloride, and mixtures thereof. Generally, the effluent coming directly out of the hydrochlorination reactor(s) shall contain a mixture of the abovementioned compounds. This first effluent shall undergo at least one downstream processing step such as a chemical or physical treatment, a separation, a storage. If a separation step is performed, the first effluent may optionally be divided into at least two effluents, which may also each constitute an effluent of the reaction step according to the invention.

As used therein, the term “downstream processing equipment” refers to any device used for processing the one or more effluent(s) of the reaction step, including for example vessels of any kinds, reactors, separators (including for example stripping vessels, distillation columns, extraction units, filtration devices, flashes, evaporators, centrifuges, agitators), condensers, tubes, pipes, heat exchangers, storage tanks, pumps, compressors, valves, flanges as well as any internal element used within such devices such as column packings, and any other equipment or connectors required to process the products from the outlet of the hydrochlorination reactor(s) to the departure of the chlorohydrin from the site of the process or its consumption in another process.

According to a second embodiment of the invention, in the downstream processing step the water is removed substantially from the effluents of the reaction step. By minimizing the concentration of water in said effluents, the corrosion of non-resistant materials in the downstream processing equipment is reduced. Any method can be employed to remove the water present in the effluents, including for example any reactive, cryogenic, extractive, azeotropic, absorptive or evaporative in-situ or ex-situ technique or any known technique for water removal.

According to a third embodiment of the invention, in the downstream processing step the concentration of hydrogen chloride in the effluents of the reaction step is reduced to below 0.8% by weight, corresponding to an aqueous pH of greater than 0.66, or to a level where non-resistant materials of construction may be employed. The treatments used therefore include, but are not limited to, dilution, neutralization, stripping, extraction, absorption, and distillation.

According to a fourth embodiment of the invention, the total fluoride concentration in each process stream or feed stream is limited to less than 50 ppm by weight, preferably less than 10 ppm by weight, more preferably less than 5 ppm by weight and most preferably less than 2 ppm by weight.

Fluoride may enter the process as a contaminant in the hydrogen chloride source employed, a contaminant of the multihydroxylated-aliphatic hydrocarbon and/or an ester thereof source employed, or as a contaminant in other process materials, such as water or inerting gases. According to the fourth embodiment of the invention, hydrogen chloride, multihydroxylated-aliphatic hydrocarbon and/or an ester thereof, or other process materials employed should contain a level of fluoride below that which compromises the stability of the materials of construction employed in the hydrochlorination process, in the hydrochlorination itself, and both upstream and downstream of the reactor. It should be appreciated that the fluoride concentration may be increased locally in parts of the process by processes such as distillation, flashing and extraction, and consequently result in localized corrosive processes. Anyway, according to this embodiment, procedures that locally concentrate fluoride in parts of the process should be avoided, or steps taken to mitigate the effects of a higher localized concentration of fluoride on the materials of construction.

According to this embodiment, when the total fluoride concentration in a process stream or feed stream is above 50 ppm by weight, preferably above 10 ppm by weight, more preferably above 5 ppm by weight and most preferably above 2 ppm by weight, such process stream or feed stream is treated to reduce the fluoride concentration to a level where the integrity of the materials of construction is not compromised. In particular, such process stream or feed stream may be treated using a fluoride scavenging agent, of a heterogeneous or of a homogenous nature. For example a sacrificial glass plate, column or tube may be employed. Other potential fluoride scavenging agents can optionally be added to the process either in a pre-treatment step or throughout the process in situ. These may include sacrificial glass beads or packed silica gel beds. Alternatively the silica gels used may be calcined spherical or cylindrical pellets such as those fabricated for use as a heterogeneous catalyst support. A wide variety of surface areas for the silica supports are possible by fabricating different mesh sizes of silica, as is known to one skilled in the art. Heterogeneous scavenging agents such as these are preferred for industrial processes for removing trace levels of fluoride. However, it is conceivable to use fluoride scavengers which are of a homogenous nature as well. These could includes sacrificial reagents such as hexamethylsiloxane, methyltrimethoxysilane or any soluble or partially soluble silicon reagent which contains a silicon-oxygen bond.

According to a preferred embodiment of the present invention, the reaction step is carried out at superatmospheric partial pressure of hydrogen chloride.

According to another preferred embodiment, the reaction step is performed with the substantial absence of water removal.

According to a particularly preferred embodiment of the invention, the reaction step is carried out at superatmospheric partial pressure of hydrogen chloride and with the substantial absence of water removal.

“Substantial absence of water removal” herein means that during the hydrochlorination reaction step or steps, no method is employed to remove the water present in the process (for example, either water of reaction or that introduced with the feed component(s)) during the hydrochlorination step. These methods may include any reactive, cryogenic, extractive, azeotropic, absorptive or evaporative in-situ or ex-situ technique or any known technique for water removal.

“Superatmospheric partial pressure of hydrogen chloride” herein means that that the hydrogen chloride partial pressure is above atmospheric pressure, i.e. 15 psia or greater.

As used therein, the term “corrosion resistant material” means a material or a mixture of materials that is not affected by the hydrochlorination reaction medium, over a period of 1 year, as measured by a loss in mass of the piece of equipment, or the dissolution of at least part of at least one of the components of the material in the reaction medium processed through the equipment gives less than 10 ppm by weight of the material in the process stream. Conversely, not resistant means that there is a measurable loss in the mass of the piece of the equipment, or that dissolution of at least a part of at least one of the components of the material in the reaction medium processed through the equipment occurs over a period of one year.

According to the present invention, any material resistant to corrosion by hydrogen chloride or hydrochloric acid may be employed as corrosion resistant material. Non-limiting materials which are resistant to corrosion include those incorporated by reference form Kirk Othmer Encyclopedia of Chemical Technology, in particular those disclosed in Kirk Othmer Encyclopedia of Chemical Technology, 2^{nd} Edition, John Wiley and Sons, publishers, 1966 volume 11 and Kirk Othmer Encyclopedia of Chemical Technology, 3^{rd} Edition, John Wiley and Sons, publishers, 1980, volume 12.

Suitable corrosion resistant materials include metals such as for example tantalum, zirconium, platinum, titanium, gold, silver, nickel, niobium, molybdenum and mixtures thereof.

Suitable corrosion resistant materials further include alloys containing at least one of the above-mentioned metals. Particularly suitable alloys include alloys containing nickel and molybdenum. Mention can be made particularly of the corrosion resistant metal alloys sold under the names Hastelloy™ or Hastalloy™, which are base on nickel as main ingredient, together with other ingredients, whose nature and percentage depend on the particular alloy, such as for example molybdenum, chromium, cobalt, iron, copper, manganese, titanium, zirconium, aluminum, carbon, tungsten.

Further suitable corrosion resistant materials include ceramics or metallic-ceramics, refractory materials, graphite, glass-lined materials, such as for example enameled steel.

Other suitable corrosion resistant materials include polymers, such as for example polyolefins such as polypropylene and polyethylene, fluorinated polymers such as polytetrafluoroethylene, polyvinylidenefluoride and polyperfluoropropylvinylether, polymers containing sulfur and/or aromatics such as polysulfones or polysulfides, resins such as epoxy resins, phenolic resins, vinylester resins, furan resins.

The corrosion resistant materials can be used to make the actual body of the downstream processing equipment devices which need to be protected from corrosion according to the present invention. The corrosion resistant materials can also be used by coating of the surface of such devices.

Mention may be made for example of coatings made from resins. For certain parts such as the heat exchangers, graphite, either impregnated or not, is particularly suited.

As used herein, the term “multihydroxylated-aliphatic hydrocarbon” refers to a hydrocarbon which contains at least two hydroxyl groups attached to separate saturated carbon atoms. The multihydroxylated-aliphatic hydrocarbon may contain, but not to be limited thereby, from 2 to about 60 carbon atoms.

Any single carbon of a multihydroxylated-aliphatic hydrocarbon bearing the hydroxyl (OH) functional group must possess no more than one OH group, and must be sp3 hybridized. The carbon atom bearing the OH group may be primary, secondary or tertiary. The multihydroxylated-aliphatic hydrocarbon used in the present invention must contain at least two sp3 hybridized carbons each bearing an OH group. The multihydroxylated-aliphatic hydrocarbon includes any vicinal-diol (1,2-diol) or triol (1,2,3-triol) containing hydrocarbon including higher orders of contiguous or vicinal repeat units. The definition of multihydroxylated-aliphatic hydrocarbon also includes for example one or more 1,3-1,4-, 1,5- and 1,6-diol functional groups as well. The multihydroxylated-aliphatic hydrocarbon may also be a polymer such as polyvinylalcohol. Geminal-diols, for example, would be precluded from this class of multihydroxylated-aliphatic hydrocarbon compounds.

It is to be understood that the multihydroxylated-aliphatic hydrocarbon can contain aromatic moieties or heteroatoms including for example halide, sulfur, phosphorus, nitrogen, oxygen, silicon, and boron heteroatoms; and mixtures thereof.

“Chlorohydrin” is used herein to describe a compound containing at least one hydroxyl group and at least one chlorine atom attached to separate saturated carbon atoms. A chlorohydrin that contains at least two hydroxyl groups is also a multihydroxylated-aliphatic hydrocarbon. Accordingly, the starting material and product of the present invention can each be chlorohydrins; in that case, the product chlorohydrin is more highly chlorinated than the starting chlorohydrin, i.e., has more chlorine atoms and fewer hydroxyl groups than the starting chlorohydrin. A preferred chlorohydrin is a highly chlorinated chlorohydrin such as a dichlorohydrin. Particularly preferred chlorohydrins are 1,3-dichloro-propan-2-ol and 2,3-dichloropropan-1-ol, and mixtures thereof.

Multihydroxylated-aliphatic hydrocarbons useful in the present invention include for example 1,2-ethanediol; 1,2-propanediol; 1,3-propanediol; 1-chloro-2,3-propanediol; 2-chloro-1,3-propanediol; 1,4-butanediol; 1,5-pentanediol; cyclohexanediols; 1,2-butanediol; 1,2-cyclohexanedimethanol; 1,2,3-propanetriol (also known as, and used herein interchangeable as, “glycerin”, “glycerine”, or “glycerol”); and mixtures thereof. Preferably, the multihydroxylated-aliphatic hydrocarbons used in the present invention include for example 1,2-ethanediol; 1,2-propanediol; 1,3-propanediol; and 1,2,3-propanetriol; with 1,2,3-propanetriol being most preferred.

Examples of esters of multihydroxylated-aliphatic hydrocarbons useful in the present invention include for example ethylene glycol monoacetate, propanediol monoacetates, glycerin monoacetates, glycerin monostearates, glycerin diacetates, and mixtures thereof. In one embodiment, such esters can be made from mixtures of multihydroxylated-aliphatic hydrocarbons with exhaustively esterified multihydroxylated-aliphatic hydrocarbons, for example mixtures of glycerol triacetate and glycerol.

The multihydroxylated-aliphatic hydrocarbons of the present invention may be used in any desirable non-limiting concentration. In general, higher concentrations are preferred for economic reasons. Useful concentrations for the present invention may include, for example from about 0.01 mole % to about 99.99 mole %, preferably from about 1 mole % to about 99.5 mole %, more preferably from about 5 mole % to about 99 mole %, and most preferably from about 10 mole % to about 95 mole %.

The hydrogen chloride source used in the present invention is preferably introduced as a gas, a liquid or in a solution or a mixture, or a mixture thereof, such as for example a mixture of hydrogen chloride and nitrogen gas, so long as the required partial pressures of the hydrogen chloride are provided for the process of the present invention.

The most preferred hydrogen chloride source is hydrogen chloride gas. Other forms of chloride may be employed in the present invention provided that the required partial pressure of hydrogen chloride is generated. Chloride in particular may be introduced with any number of cations including those associated with phase transfer reagents such as quaternary ammonium and phosphonium salts (for example tetra-butylphosphonium chloride). Alternatively, ionic liquids such n-butyl-2-methylimidazolium chloride may be used as a synergist to promote the acid catalyzed displacement of OH from the multihydroxylated-aliphatic hydrocarbon.

It is also known that these other halide sources may act as co-catalysts for the hydrochlorination of alcohols. In this respect catalytic amounts of iodide or bromide may be used to accelerate these reactions. These reagents may be introduced as gases, liquids or as counterion salts using a phase transfer or ionic liquid format. The reagents may also be introduced as metal salts wherein the alkali or transition metal counterion does not promote oxidation of the multihydroxylated-aliphatic hydrocarbon. Care must be employed in using these co-catalysts in controlled hydrochlorination processes because the potential for RCl formation may increase. Mixtures of different sources of halide may be employed, for example hydrogen chloride gas and an ionic chloride, such as tetraalkylammonium chloride or a metal halide. For example, the metal halide may be sodium chloride, potassium iodide, potassium bromide and the like.

In an embodiment of the present invention where the multihydroxylated-aliphatic hydrocarbon is the starting material, as opposed to an ester of the multi-hydroxylated aliphatic hydrocarbon as a starting material, it is preferred that the formation of chlorohydrin be promoted by the presence of a catalyst. In another embodiment of the present invention, where the ester of the multihydroxylated-aliphatic hydrocarbon is used as the starting material, preferably a partial ester, the catalyst exists inherently in the ester, and therefore the use of a separate catalyst component is optional. However, an additional catalyst may still be included in the present process to further promote conversion to the desired products. Additional catalyst may also be used in the case where the starting material includes a combination of esterified and nonesterifed multihydroxylated-aliphatic hydrocarbons.

According to an embodiment of the invention, a catalyst is used in the reaction step of the process of the present invention, the catalyst may be for example a carboxylic acid; an anhydride; an acid chloride; an ester; a lactone; a lactam; an amide; a metal organic compound such as sodium acetate; or a combination thereof. Any compound that is convertible to a carboxylic acid or a functionalized carboxylic acid under the reaction conditions of the present invention may also be used.

A preferred carboxylic acid is an acid with a functional group consisting of a halogen, an amine, an alcohol, an alkylated amine, a sulfhydryl, an aryl group or an alkyl group, or combinations thereof, wherein this moiety is not sterically hindering the carboxylic acid group. A preferred acid for this present process is acetic acid.

Examples of carboxylic acids usefulness as a catalyst in the present invention include, acetic acid, propionic acid, 4-methylvaleric acid, adipic acid, 4-droxyphenylacetic acid, 6-chlorohexanoic acid, 4-aminobutyric acid, hexanoic acid, heptanoic acid, 4-dimethylaminobutyric acid, 6-aminohexanoic acid, 6-hydroxyhexanoic acid, 4-aminophenylacetic acid, 4-trimethylammonium butyric acid chloride, polyacrylic acid, polyethylene grafted with acrylic acid, a divinylbenzene/methacrylic acid copolymer, and mixtures thereof. Examples of anhydrides include acetic anhydride, maleic anhydride, and mixtures thereof. Examples of acid chlorides include acetyl chloride, 6-chlorohexanoyl chloride, 6-hydroxyhexanoyl chloride and mixtures thereof. Examples of esters include methyl acetate, methyl propionate, methyl pivalate, methyl butyrate, ethylene glycol monoacetate, ethylene glycol diacetate, propanediol monoacetates, propanediol diacetates, glycerin monoacetates, glycerin diacetates, glycerin triacetate, a glycerin ester of a carboxylic acid (including glycerin mono-, di-, and tri-esters), and combinations thereof. Examples of most preferred lactones include ε-caprolactone, γ-butyrolactone, δ-valerolactone and mixtures thereof. An example of a lactam is ε-caprolactam. Zinc acetate is an example of a metal organic compound.

A preferred catalyst used in the present invention is a carboxylic acid, an ester of a carboxylic acid, or a combination thereof, particularly an ester or acid having a boiling point higher than that of the desired highest boiling chlorohydrin that is formed in the reaction mixture so that the chlorohydrin can be removed without removing the catalyst. Catalysts which meet this definition and are useful in the present invention include for example, polyacrylic acid, glycerin esters of carboxylic acids (including glycerin mono-, di-, and tri-esters), polyethylene grafted with acrylic acid, 6-chlorohexanoic acid, 4-chlorobutanoic acid, caprolactone, heptanoic acid, 4-hydroxyphenylacetic acid, 4-aminophenylacetic acid, 6-hydroxyhexanoic acid, 4-aminobutyric acid, 4-trimethylammoniumbutyric acid chloride, stearic acid, 5-chlorovaleric acid, 6-hydroxyhexanoic acid, 4-aminophenylacetic acid, and mixtures thereof.

Carboxylic acids of formula RCOOH catalyze the hydrochlorination of multihydroxylated-aliphatic hydrocarbons to chlorohydrins. The specific carboxylic acid catalyst chosen for the process of the present invention may be based upon a number of factors including for example, its efficacy as a catalyst, its corrosiveness, its cost, its stability to reaction conditions, and its physical properties. The particular process, and process scheme in which the catalyst is to be employed may also be a factor in selecting the particular catalyst for the present process. The “R” groups of the carboxylic acid may be chosen from hydrogen or hydrocarbyl groups, including alkyl, aryl, aralkyl, and alkaryl. The hydrocarbyl groups may be linear, branched or cyclic, and may be substituted or un-substituted. Permissible substituents include any functional group that does not detrimentally interfere with the performance of the catalyst, and may include heteroatoms. Non-limiting examples of permissible functional groups include chloride, bromide, iodide, hydroxyl, phenol, ether, amide, primary amine, secondary amine, tertiary amine, quaternary ammonium, sulfonate, sulfonic acid, phosphonate, and phosphonic acid.

The carboxylic acids useful in the present invention may be monobasic such as acetic acid, formic acid, propionic acid, isobutyric acid, hexanoic acid, heptanoic acid, oleic acid, or stearic acid; or polybasic such as succinic acid, adipic acid, or terephthalic acid. Examples of aralkyl carboxylic acids include phenylacetic acid and 4-aminophenylacetic acid. Examples of substituted carboxylic acids include 4-aminobutyric acid, 4-dimethylaminobutyric acid, 6-aminocaproic acid, 4-aminophenylacetic acid, 4-hydroxyphenylacetic acid, lactic acid, glycolic acid, 4-dimethylaminobutyric acid, and 4-trimethylammoniumbutyric acid. Additionally, materials that can be converted into carboxylic acids under reaction conditions, including for example carboxylic acid halides, such as acetyl chloride; carboxylic acid anhydrides such as acetic anhydride; carboxylic acid esters such as methyl acetate; multihydroxylated-aliphatic hydrocarbon acetates such as glycerol 1,2-diacetate; carboxylic acid amides such as ε-caprolactam and γ-butyrolactam; and carboxylic acid lactones such as γ-butyrolactone, δ-valerolactone and ε-caprolactone may also be employed in the present invention. Mixtures of carboxylic acids may also be used in the present invention.

Some carboxylic acid catalysts that may be used in the present invention are less effective than others in the hydrochlorination process of the present invention, such as those bearing sterically demanding substituents close to the carboxylic acid group, for example 2,2-dimethylbutyric acid, sterically hindered 2-substituted benzoic acids such as 2-aminobenzoic acid and 2-methylaminobenzoic acid. For this reason, carboxylic acids that are sterically unencumbered around the carboxylic acid group are more preferred.

In the process of the present invention, preferred acid catalysts used in the present invention include for example acetic acid, propionic acid, butyric acid, isobutyric acid, hexanoic acid, heptanoic acid, 4-hydroxyphenylacetic acid, 4-aminophenylacetic acid, 4-aminobutyric acid, 4-dimethylaminobutyric acid, 4-trimethylammonium butyric acid chloride, succinic acid, 6-chlorohexanoic acid, 6-hydroxyhexanoic acid, and mixtures thereof.

According to another aspect of the present invention, the reaction step in which the multihydroxylated-aliphatic hydrocarbon or ester thereof is contacted with hydrogen chloride under reaction conditions to produce the chlorohydrin or ester thereof, is performed in the presence of a catalyst, wherein said catalyst (i) is a carboxylate derivative having from two to about 20 carbon atoms and containing at least one functional group selected from the group comprising an amine, an alcohol, a halogen, an sulfhydryl, an ether, an ester, or a combination thereof, wherein the functional group is attached no closer to the acid function than the alpha carbon; or a precursor thereto; (ii) is less volatile than the chlorohydrin, ester of a chlorohydrin, or a mixture thereof; and (iii) contains heteroatom substituents.

Within this aspect of the present invention, one embodiment of the catalyst structure of the present invention is generally represented by Formula (a) shown below wherein the functional group “R′” includes a functional group comprising an amine, an alcohol, a halogen, a sulfhydryl, an ether, an ester, or an alkyl, an aryl or alkaryl group of from 1 to about 20 carbon atoms containing said functional group; or a combination thereof; and wherein the functional group “R” may include a hydrogen, an alkali, an alkali earth or a transition metal or a hydrocarbon functional group.

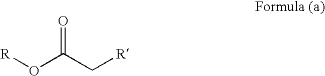

In accordance with this aspect of the present invention, the certain catalysts may also be advantageously employed at superatmospheric, atmospheric or sub-atmospheric pressure, and particularly in circumstances where water is continuously or periodically removed from the reaction mixture to drive conversion to desirably higher levels. For example, the hydrochlorination of glycerol reaction can be practiced by sparging hydrogen chloride gas through a mixture of a multihydroxylated-aliphatic hydrocarbon and a catalyst. In such a process, a volatile catalyst, such as acetic acid, may be at least partially removed from the reaction solution by the hydrogen chloride gas being sparged through the solution and may be lost from the reaction medium. The conversion of the multihydroxylated-aliphatic hydrocarbon to desired chlorohydrins may consequently be slowed because the catalyst concentration is reduced. In such a process, the use of less volatile catalysts, such as 6-hydroxyhexanoic acid, 4-aminobutyric acid; dimethyl 4-aminobutyric acid; 6-chlorohexanoic acid; caprolactone; carboxylic acid amides such as e-caprolactam and γ-butyrolactam; carboxylic acid lactones such as γ-butyrolactone, δ-valerolactone and ε-caprolactone; caprolactam; 4-hydroxyphenyl acetic acid; 6-aminocaproic acid; 4-aminophenylacetic acid; lactic acid; glycolic acid; 4-dimethylaminobutyric acid; 4-trimethylammoniumbutyric acid; and combination thereof; and the like may be preferred. It is most desirable to employ a catalyst, under these atmospheric or subatmospheric conditions, that is less volatile than the desired chlorohydrin being produced. Furthermore, it is desirable that the catalyst be fully miscible, with the multihydroxylated-aliphatic hydrocarbon employed. If the catalyst is not fully miscible, it may form a second phase and the full catalytic effect may not be realized. For this reason, it may be desirable that the catalyst contain polar heteroatom substituents such as hydroxyl, amino or substituted amino, or halide groups, which render the catalyst miscible with the multihydroxylated-aliphatic hydrocarbon, for example, glycerol.

The choice of a catalyst, for example a carboxylic acid catalyst, for use in the process of the present invention may also be governed by the specific process scheme employed for multihydroxylated-aliphatic hydrocarbon hydrochlorination. For example, in a once-through process where a multihydroxylated-aliphatic hydrocarbon is reacted to as high a conversion as possible to the desired chlorohydrin, which then is further converted to other products without separation from the catalyst, the carboxylic acid catalyst is subsequently not utilized further. In such a process scheme, it is desirable that the carboxylic acid be inexpensive, in addition to being effective. A preferred carboxylic acid catalyst in such a situation would be for example acetic acid.

In a recycle process, for example, wherein the produced chlorohydrins are separated from the carboxylic acid catalyst before further processing or use, the carboxylic acid catalyst is additionally chosen based on the ease of separation of the catalyst, and its esters with the reaction products, from the desired chlorohydrin products. In such a case, it may be preferable to employ a heavy (i.e. lower volatility) acid so that it can be readily recycled to the reactor with unreacted glycerol or intermediate monochlorohydrins for further reaction. Suitable heavy acids useful in the present invention include for example 4-hydroxyphenylacetic acid, heptanoic acid, 4-aminobutyric acid, caprolactone, 6-hydroxyhexanoic acid, 6-chlorohexanoic acid, 4-dimethylaminobutyric acid, 4-trimethylammoniumbutyric acid chloride, and mixtures thereof.

It is also preferred that the acid, or its esters with the multihydroxylated-aliphatic hydrocarbon being hydrochlorinated, or its esters with the reaction intermediates or reaction products be miscible in the reaction solution. For this reason it may be desirable to select the carboxylic acid catalyst taking these solubility constraints into consideration. Thus, for example, if the multihydroxylated-aliphatic hydrocarbon being hydrochlorinated is very polar, such as glycerol, some carboxylic acid catalysts would exhibit less than complete solubility, and would form two phases upon mixing. In such a case, a more miscible acid catalyst, such as acetic acid or 4-aminobutyric acid may be desirable.

The catalysts useful in the present invention are effective over a broad range of concentrations, for example from about 0.01 mole % to about 99.9 mol % based upon the moles of multihydroxylated-aliphatic hydrocarbon, preferably from about 0.1 mole % to about 67 mole %, more preferably from about 0.5 mole % to about 50 mole % and most preferably from about 1 mole % to about 40 mole %. The specific concentration of catalyst employed in the present invention may depend upon the specific catalyst employed in the present invention and the process scheme in which such catalyst is employed.

For example, in a once-through process where the catalyst is used only once and then discarded, it is preferred to employ a low concentration of a highly active catalyst. In addition, it may be desirable to employ an inexpensive catalyst. In such a process, concentrations of for example, from about 0.01 mole % to about 10 mole % based on the multihydroxylated-aliphatic hydrocarbon may be used, preferably from about 0.1 mole % to about 6 mole %, more preferably from about 1 mole % to about 5 mole %.

In process schemes, for example, where the catalyst is recycled and used repeatedly, it may be desirable to employ higher concentrations than with a catalyst that is discarded. Such recycled catalysts may be used from about 1 mole % to about 99.9 mole % based on the multihydroxylated-aliphatic hydrocarbon, preferably from about 5 mole % to about 70 mole %, more preferably from about 5 mole % to about 50 mole %, although these concentrations are to be considered non-limiting. Higher catalysts concentrations may be desirably employed to reduce the reaction time, minimize the size of process equipment and reduce the formation of undesirable, uncatalyzed side products.

According to a preferred embodiment of the invention, the hydrochlorination reaction step of the process of the present invention is carried out under superatmospheric pressure conditions. “Superatmospheric pressure” herein means that the hydrogen chloride (HCl) partial pressure is above atmospheric pressure, i.e. 15 psia or greater. Generally, the hydrogen chloride partial pressure employed in the reaction step of the process of the present invention is at least about 15 psia or greater. Preferably, the hydrogen chloride partial pressure of the reaction step of the present process is not less than about 25 psia, more preferably not less than about 35 psia HCl, and most preferably not less than about 55 psia; and preferably not greater than about 1000 psia, more preferably not greater than about 600 psia, and most preferably not greater than about 150 psia.

The hydrogen chloride used in the present invention is most preferably anhydrous. The hydrogen chloride composition can range from 100 volume % hydrogen chloride to about 50 volume % hydrogen chloride. Preferably, the hydrogen chloride feed composition is greater than about 50 volume % hydrogen chloride, more preferably greater than about 90 volume % hydrogen chloride, and most preferably greater than about 99 volume % hydrogen chloride.

The temperatures useful in the practice of the reaction step of the process of the present invention are sufficient to give economical reaction rates, but not so high that starting material, product or catalyst stability become compromised. Furthermore, high temperatures increase the rate of undesirable uncatalyzed reactions, such as non-selective over-chlorination, and can result in increased rates of equipment corrosion. Useful temperatures in the present invention generally may be from about 25° C. to about 300° C., preferably from about 25° C. to about 200° C., more preferably from about 30° C. to about 160° C., even more preferably from about 40° C. to about 150° C., and most preferably from about 50° C. to about 140° C.

The reaction of the superatmospheric pressure process of the present invention is advantageously rapid and may be carried out for a time period of less than about 12 hours, preferably less than about 5 hours, more preferably less than about 3 hours and most preferably less than about 2 hours. At longer reaction times, such as above about 12 hours, the process begins to form RCls and other over-chlorinated by-products.

Surprisingly, it has been discovered that high per-pass yields and high selectivity can be achieved using the superatmospheric pressure process of the present invention. For example, a per-pass yield for the chlorohydrin based on the multihydroxylated-aliphatic hydrocarbon of greater than about 80%, preferably greater than about 85%, more preferably greater than about 90%, and most preferably greater than about 93% can be achieved by the present invention. For example, a high selectivity of greater than about 80%, preferably greater than about 85%, more preferably greater than about 90%, and most preferably greater than about 93% of chlorohydrins can be achieved by the process of the present invention. Of course, yields can be increased by recycling reaction intermediates.

For example, when the multihydroxylated-aliphatic hydrocarbon used in the present invention is glycerol, recycling intermediate monochlorohydrins can increase the ultimate yield of dichlorohydrins achieved. Moreover, unlike many of the processes of the prior art, water removal is not an essential feature of the process of the present invention in carrying out the reaction which forms the chlorohydrins. In fact, the reaction of the present invention is preferentially carried out in the absence of water removal such as azeotropic removal of water.

In the superatmospheric pressure process of the present invention, it is also not necessary to use starting materials that are free of contaminants such as water, salts or organic impurities other than multihydroxylated-aliphatic hydrocarbons. Accordingly, the starting materials may contain, generally, no more than about 50 weight percent of such contaminants. For example, crude 1,2,3-propanetriol (crude glycerol) that may contain water (from about 5% to about 25% weight percent), alkali (for example, sodium or potassium) or alkaline earth (for example, calcium or magnesium) metal salts (from about 1% to about 20% by weight), and/or alkali carboxylate salts (from about 1% to about 5% by weight), can also be used in the present invention effectively to produce the desired product. Consequently, the process of the present invention is a particularly economical approach.

In one embodiment of the process of the present invention, 1,2,3-propanetriol (glycerol) is placed in a closed vessel, and heated and pressurized under an atmosphere of hydrogen chloride gas in the presence of the aforementioned catalytic amount of a carboxylic acid or ester thereof. Under the preferred conditions of the process, the major product is 1,3-dichloropropan-2-ol (for example, more than 90% yield), with minor amounts (for example, less than 10% total yield) of the following products: 1-chloro-2,3-propanediol, 2-chloro-1,3-propanediol and 2,3-dichloropropan-1-ol; and no detectable amounts (less than 200 ppm) of 1,2,3-trichloropropane. Advantageously, both the major and minor dichlorinated products (1,3-dichloro-propan-2-ol and 2,3-dichloropropan-1-ol) are precursors to epichlorohydrin. The dichlorinated products can readily be converted to epichlorohydrin by reaction with base, as is well-known in the art.

The present invention may include various process schemes, including for example batch, semi-batch, or continuous.

The multihydroxylated-aliphatic hydrocarbon may be employed neat or diluted in an appropriate solvent. Such solvents may include for example water and alcohols. It may be preferred to purify the multihydroxylated-aliphatic hydrocarbon before it is employed in the hydrochlorination reaction by removing contaminants, including water, organic materials or inorganic materials before use. This purification may include well known purification techniques such as distillation, extraction, absorption, centrifugation, or other appropriate methods. The multihydroxylated-aliphatic hydrocarbon is generally fed to the process as a liquid although this is not absolutely necessary.

The hydrogen chloride employed in the process is preferably gaseous. The hydrogen chloride may, however, be diluted in a solvent such as an alcohol (for example methanol); or in a carrier gas such as nitrogen, if desired. Optionally, the hydrogen chloride may be purified before use to remove any undesirable contaminants. It is preferred that the hydrogen chloride be substantially anhydrous although some amounts (for example less than about 50 mole %, preferably less than about 20 mole %, more preferably less than about 10 mole %, even more preferably less than about 5 mole %, most preferably less than about 3 mole %) of water present in the hydrogen chloride are not excessively detrimental. The hydrogen chloride is fed to the process equipment in any suitable manner. It is preferred that the process equipment is designed to ensure good dispersal of the hydrogen chloride throughout the hydrochlorination reactor that is employed in the present process. Therefore, single or multiple spargers, baffles and efficient stirring mechanisms are desirable.

The catalyst employed may be fed to the process equipment independently, or as a mixture with, or component of, the multihydroxylated-aliphatic hydrocarbon or hydrogen chloride feeds.

The equipment useful for the hydrochlorination reaction step of the present invention may be any well-known equipment in the art and should be capable of containing the reaction mixture at the conditions of the hydrochlorination.

According to a preferred embodiment of the present invention, the equipment used to perform the reaction step is at least partially made of or covered with corrosion resistant material as described above. According to a particularly preferred embodiment of the present invention, the equipment used to perform the reaction step is totally made of or covered with corrosion resistant material as described above.

In an exemplifying batch process, the multihydroxylated aliphatic hydrocarbon and hydrochlorination catalyst are charged to a reactor. Hydrogen chloride is then added to the desired pressure and the reactor contents heated to the desired temperature for the desired length of time. The reactor contents are then discharged from the reactor and undergo at least one downstream processing step such as for example separation, purification and/or storage.

In an illustrative semi-batch process, one or more of the reagents is fed to a reactor over a period of time throughout the reaction while other reagents are fed only at the start of the reaction. In such a process, for example, the multihydroxylated-aliphatic hydrocarbon and catalyst may be fed in a single batch to a hydrochlorination reactor, which is then held at reaction conditions for a suitable time, while hydrogen chloride is fed continuously throughout the reaction at the desired rate, which may be at constant flow, or constant pressure. After the reaction, the hydrogen chloride feed can be terminated and the reactor contents may be discharged at least one downstream processing step such as for example separation, purification and/or storage.

In the large-scale production of chemicals it is often desirable to employ a continuous process since the economic advantage of doing so is usually greater than for batch processing. The continuous process may be, for example, a single-pass or a recycle process. In a single-pass process, one or more of the reagents pass through the process equipment once, and then the resulting effluent from the reactor is sent for downstream processing such as for example separation, purification and/or storage. In such a scheme, the multihydroxylated-aliphatic hydrocarbon and catalyst may be fed to the equipment and hydrogen chloride added as desired at a single point or at multiple points throughout the process equipment, which may include continuous stirred tank reactors, tubes, pipes or combinations thereof.

Alternatively, the catalyst employed may be a solid which is retained within the process equipment by means of a filter or equivalent device. The reagents and catalysts are fed at such a rate that the residence time in the process equipment is appropriate to achieve a desired conversion of the multihydroxylated-aliphatic hydrocarbon to products. The material exiting the process equipment is sent to downstream processing such as for example separation, purification and/or storage. In such a process, it is generally desirable to convert as much multihydroxylated-aliphatic hydrocarbon to desired product as possible.

In a continuous recycle process, one or more of the unreacted multihydroxylated-aliphatic hydrocarbon, reaction intermediates, hydrogen chloride, or catalyst exiting from the process equipment are recycled back to a point earlier in the process. In this manner, raw material efficiencies are maximized or catalysts reused. Since catalysts are reused in such a process scheme, it may be desirable to employ the catalysts in a higher concentration than they are employed in a single-pass process where they are often discarded. This may result in faster reactions, or smaller process equipment, which results in lower capital costs for the equipment employed.

Removal of the desired product from the catalysts or other process components can be achieved in a variety of ways. It may be possible to achieve the separation, for example, by vaporization in a continuous fashion, either directly from the hydrochlorination reactor, or a separate piece of equipment such as a vaporizer or a distillation column. In such a case, a catalyst that is less volatile than the desired product would be employed, so that the catalyst is retained within the process equipment. Alternatively, a solid catalyst may be employed, and the separation may be achieved, for example, by filtration, centrifugation or vaporization. Liquid extraction, absorption or chemical reaction may also be employed in some cases to recycle catalysts or reaction intermediates.

In one embodiment of the present invention, a multihydroxylated-aliphatic hydrocarbon is hydrochlorinated using a hydrochlorination catalyst chosen to be less volatile than the desired hydrochlorination products. After the hydrochlorination reaction, additional multihydroxylated-aliphatic hydrocarbon is added to the reaction products, excess starting materials, reaction intermediates and catalyst. It is thought that this liberates some of the desired hydrochlorination product which may have existed as an ester of the catalyst, so that the desired product can be more completely recovered from the reaction solution by vaporization. After recovery of the desired hydrochlorination product, the remainder of the process stream can be recycled to the hydrochlorination stream. This process scheme also may have the advantage of minimizing the amount of hydrogen chloride lost since much of that remaining in the process stream after addition of multihydroxylated-aliphatic hydrocarbon would be consumed by reaction with the newly added multihydroxylated-aliphatic hydrocarbon.

The particular process scheme employed may depend upon many factors including, for example, the identity, cost and purity of the multihydroxylated-aliphatic hydrocarbon being hydrochlorinated, the specific process conditions employed, the separations required to purify the product, and other factors. The examples of processes described herein are not to be considered as limiting the present invention.

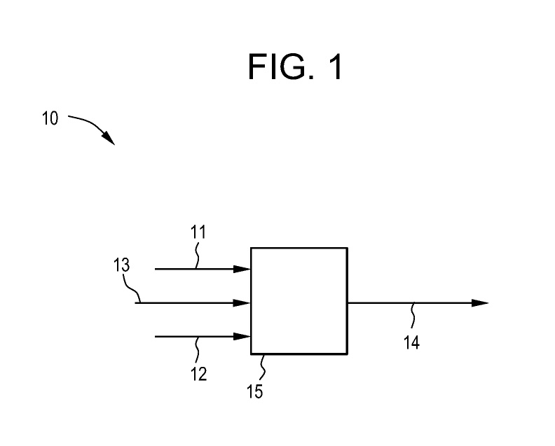

FIGS. 1, 2 and 3 show three non-limiting embodiments of the hydrochlorinated process of the present invention. The examples illustrating the present invention process shown in FIGS. 1, 2 and 3 are only preferred embodiments of the present invention.

FIG. 1, for example, shows a process of the present invention generally indicated by numeral 10, wherein a multihydroxylated-aliphatic hydrocarbon such as a glycerol feed stream, 11, is introduced into a reaction vessel, 15. The reaction vessel 15, may be of any well-known suitable type, including for example, one or more continuous stirred tank reactors (CSTRs) or tubular reactors; or combinations thereof.

Also introduced to vessel 15, are a hydrogen chloride feed stream, 12, and a carboxylic acid or carboxylic acid precursor catalyst feed stream, 13. Streams 12 and 13 may be introduced into vessel 15 either separately or together. In addition, optionally, all of the streams 11, 12, and 13 may be combined together into one feed stream. Any of the streams 11, 12, or 13, may be introduced at a single point or at multiple points of vessel 15. In vessel 15, glycerol is partially or fully converted to its esters with the carboxylic acid catalyst, monochlorohydrins and dichlorohydrins and their esters. The effluents of the reaction step, containing, for example dichlorohydrins, monochlorohydrins, unreacted glycerol, and their esters, water, unreacted hydrogen chloride and catalyst exits vessel 15 as stream 14, are then sent to a downstream processing equipment such as storage, separation, purification, and then optionally to other equipment for further reaction such as for example a reaction with a base to form epichlorohydrin.

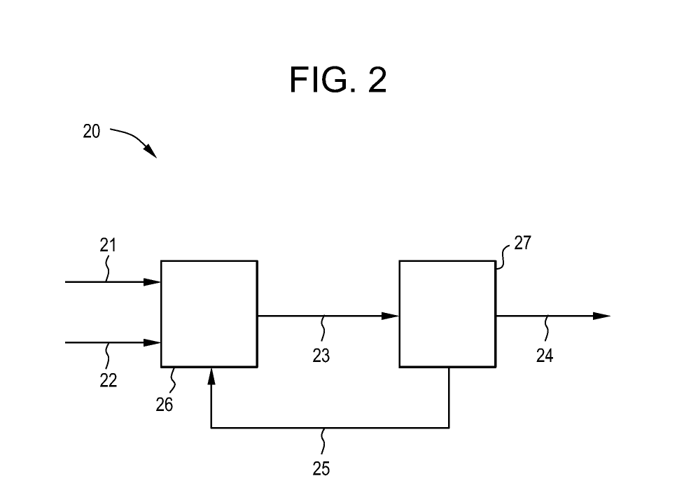

FIG. 2 shows another embodiment of the process of the present invention generally indicated by numeral 20, in which a feed stream 21 containing a multihydroxylated-aliphatic hydrocarbon such as a glycerol is fed to reaction vessel 26, which may be one or more CSTRs or tubular reactors, or combinations thereof. Also fed to vessel 26 is feed stream 22, containing hydrogen chloride. Also fed to vessel 26 is a recycle stream 25, recycled from vessel 27, containing, for example, unreacted glycerol, monochlorohydrins and their esters with the catalyst, which is also recycled in this stream 25.

In the reaction vessel 26, glycerol is converted to monochlorohydrins and their esters; and monochlorohydrins are converted to dichlorohydrins and their esters. Stream 23, containing, for example, dichlorohydrins, monochlorohydrins, unreacted glycerol and their esters with the carboxylic acid catalyst, water, unreacted hydrogen chloride and catalyst exits vessel 26, and is fed to downstream processing vessel 27. In vessel 27, at least some of the desired dichlorohydrins, water, and unreacted hydrogen chloride, as stream 24, are separated from monochlorohydrins and their esters, unreacted glycerol and its esters and catalyst, as recycle stream 25, which is recycled to vessel 26. Stream 25 may also optionally contain some remaining dichlorohydrins and their esters.

Vessel 27 may comprise any well-known suitable separation vessel, including one or more distillation columns, flash vessels, extraction or absorption columns, or any suitable known separation apparatuses known in the art. According to the invention, in vessel 27 the effluent containing the chlorohydrins and/or esters thereof is kept at a temperature of less than 120° C.

Product stream 24 may then be sent to storage, to further processing for example purification, provided it is kept at a temperature of less than 120° C.

Product stream 24 may also be sent to a further reaction, for example, conversion to epichlorohydrin. In one example of this process scheme, the catalyst may be chosen such that its chemical or physical properties result in a ready separation of the catalyst or its esters from the desired dichlorohydrins. For example, the catalyst selected for this process scheme may be 6-chlorohexanoic acid, caprolactone, 4-chlorobutyric acid, stearic acid, or 4-hydroxyphenylacetic acid.

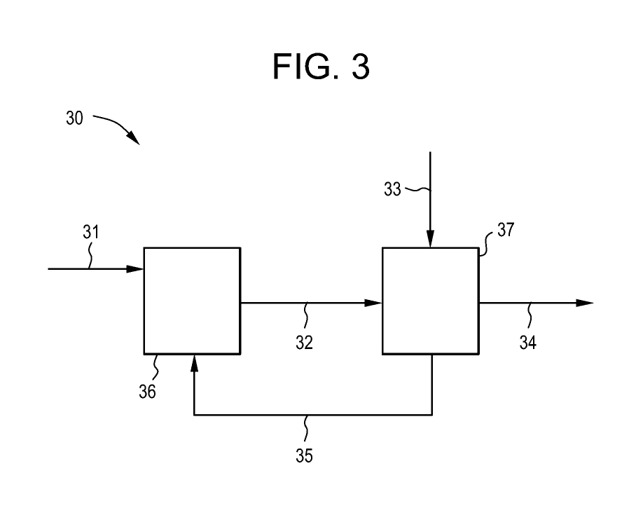

FIG. 3 shows another embodiment of the process of the present invention generally indicated by numeral 30, in which a reaction vessel 36 is fed with a feed stream 31, containing hydrogen chloride; and a recycle stream containing glycerol, glycerol esters, monochlorohydrin and their esters and catalyst, via stream 35. In vessel 36, which may comprise one or more CSTRs, one or more tubular reactors or combinations thereof, glycerol and monochlorohydrins are converted to dichlorohydins. Stream 32, containing, for example, dichlorohydrins, monochlorohydrins, glycerol and their esters, catalyst, unreacted hydrogen chloride and water exists vessel 36 and is fed to unit 37. Also fed to unit 37 is feed stream 33, containing glycerol.

Unit 37 contains a reaction part, and a downstream processing separation part. In the reaction part of unit 37 which includes at least one reaction vessel such as, for example, a stirred tank, a tubular reactor, or a combination thereof, glycerol reacts with the esters of monochlorohydrins and dichlorohydins to substantially liberate the free monochlorohydrins and dichlorohydins and forming glycerol esters. Additionally, at least some of the unreacted hydrogen chloride that enters unit 37 via stream 32 is also consumed to form mainly monochlorohydrins. Unit 37 also serves as a means to separate the desired dichlorohydrins from unreacted monochlorohydrins and glycerol and their esters, and includes therefore at least one downstream processing equipment such as, for example, one or more distillation columns, flash vessels, extractors, or any other separation equipment.

According to the invention, in the downstream processing separation part of unit 37, the effluent containing the chlorohydrins and/or esters thereof is kept at a temperature of less than 120° C. Product stream 34, exiting unit 37 and containing dichlorohydrins, water and residual hydrogen chloride may then be sent to further processing for example purification to or storage, provided it is kept at a temperature of less than 120° C. Product stream 34 may also be sent to a process for further reaction, for example to a reaction process for preparing epichlorohydrin.

Stream 35, containing glycerol and monochlorohydrins and their esters and catalyst exits vessel 37 to be recycled, as stream 35, to the vessel 36.

In the process configuration of FIG. 3, it may be desirable to use relatively large amounts of catalyst, for example from about 10 mole % to about 70 mole % based on glycerol so that the rate of the hydrochlorination reaction in vessel 36 is very fast, and the equipment consequently small. It is also preferred that the catalyst, in the process configuration of FIG. 3, possess chemical or physical properties such that the separation in unit 37 is facilitated, for example, the use of a catalyst that boils at a temperature substantially below that at which the lowest boiling dichlorohydrins boils may be preferred when the separation method is distillation. Examples of such catalysts include 6-chlorohexanoic acid, heptanoic acid, and 4-hydroxyphenylacetic acid.

### EXPERIMENTAL

Experiments were performed in magnetically-stirred, round-bottomed glass flasks equipped with a water-cooled condenser. Unless otherwise stated, experiments were done under air. The desired amounts of reagents were mixed and stirred at room temperature for a few minutes before being sampled to determine the initial composition. The flasks were then immersed in an oil bath that had been heated to the desired temperature. Samples were taken for analysis at defined times. Casual observation of the temperature readings suggested that the bath temperatures varied by no more that ±2° C. throughout the experiments. Samples were analyzed by gas chromatography. Most chemicals were from commercial supplies. Glycerol, 1,3-dichloropropan-2-ol (1,3-dichlorohydrin, 1,3-DCH), and 3-chloropropane-1,2-diol (1-monochlorohydrin, 1-MCH) were obtained from Aldrich Chemical and caprolactone from TCI. Distilled water was employed.

“Corrosion metals” were obtained by dissolving a small piece of Hastelloy B4 in concentrated hydrochloric acid by heating to reflux until all the metal had dissolved after several days, the concentrated hydrochloric acid being replenished periodically during this time. The resulting solution was dried in a vacuum oven to yield a lustrous, deep-green solid.

### Examples 1 and 2

The following mixtures were made, which are representative of the composition of the effluents of the hydrochlorination reaction of glycerol.

Mixture #1

| | |
|---|---|
| 1,3-Dichloropropan-2-ol | 5 g |
| 1-Chloropropen-2,3-diol | 5 g |
| Glycerol | 1 g |
| Water | 1 g |

Mixture #2

| | |
|---|---|
| 1,3-Dichloropropan-2-ol | 5 g |
| 1-Chloropropen-2,3-diol | 5 g |
| Glycerol | 1 g |
| Water | 1 g |
| Caprolactone | 1 g |

Each mixture underwent the sequential heat treatment indicated below and the pH was measured using damp pH paper. The results were the same for each solution.

Incremental

| Time (hr) | Temperature (° C.) | pH |
|---|---|---|
| 0.5 | 50 | 3 |
| 0.5 | 50 | 3 |
| 0.5 | 75 | 3 |
| 0.75 | 75 | 2 |
| 0.75 | 100 | 2 |
| 0.75 | 120 | 1 |
| 0.75 | 120 | 1 |
| 0.75 | 140 | 1 |
| 0.5 | 140 | 1 |
| 0.75 | 150 | 1 |
| 0.75 | 150 | 1 |
| cooled to | 50 | 1 |

These results indicate that the acidity of the effluents of the hydrochlorination reaction increases with increasing temperature, and that once heat treated, the acidity does not decrease upon cooling.

### Examples 3-7

Weighed metal coupons were charged to a Fisher-Porter tube reactor. In some cases two coupons were charged to the same tube, and in these cases a Teflon spacer was also added to prevent contact between the dissimilar metals.
To prepare the reaction mixture for the corrosion test, glycerol and caprolactone were charged to tubes to just cover the coupons, and the equipment assembled. The atmosphere in the tubes was replaced with HCl by three pressurization/venting cycles, the HCl pressure raised to ca. 30 psi and the vessels heated to the desired temperature. When this desired temperature was reached, HCl was fed on demand at the desired final pressure. HCl from the gas phase was absorbed in the liquid phase and reacted with glycerol resulting in a reaction mixture comprising dichlorohydrins, monochlorohydrins, and their esters, water, HCl and catalyst. After the desired corrosion testing time, the reactors were depressurized, the contents discharged and the coupons washed with water, and acetone, dried and weighed to determine any loss of mass.

TABLE 1
Corrosion Tests - Conditions: 125 hrs at 130° C., 130 psig HCl
followed by 96 hrs at 25° C., 20 psig HCl.

| | Initial Mass | Final Mass | Mass Lost |
|---|---|---|---|
| Metal Coupon | (g) | (g) | (%) |
| Tantalum | 6.7791 | 6.7790 | 0.0015 |
| Tantalum KBI | 4.8983 | 4.8947 | 0.0735 |
| Zirconium | 10.9860 | 8.6585 | 21.1861 |
| Niobium | 11.3320 | 10.9417 | 3.4442 |
| Hastelloy B3 | 18.2757 | 17.8442 | 2.3611 |

During the reaction it was clear that corrosion metals from the manifold had contaminated the reaction solutions.

### Examples 8-10

The second set of experiments was done in the same manner as the first, and the 130° C. temperature, 130 psig pressure conditions were maintained for 161 hours. The purpose of the 2^{nd} experiment was essentially to compare corrosion rates of Hastelloy B and Hastelloy C with tantalum under identical conditions. Corrosion metals from the manifold again contaminated the test solutions. Results are shown in the table below.

TABLE 2
Corrosion Tests - Conditions: 161 hrs at 130° C., 130 psig HCl.

| | Initial Mass | Final Mass | Mass Lost |
|---|---|---|---|
| Metal Coupon | (g) | (g) | (%) |
| Tantalum | 6.7790 | 6.7790 | 0.0000 |
| Hastelloy C276 | 15.9879 | 15.5618 | 2.6651 |
| Hastelloy B3 | 20.1879 | 20.1212 | 0.3304 |

The results show that corrosion rate for Hastelloy B was substantially less than the corrosion rate for Hastelloy C.

### Examples 11-14

In the third set of experiments two grades of Hastelloy®, C3 and B4, were immersed in glycerol hydrochlorination reaction effluents, containing mainly dichlorohydrins, water and dissolved HCl. These reaction effluents had been made in a Hastelloy C reactor under harsh conditions and were consequently already contaminated with corrosion metals, particularly nickel chloride. The effluents containing the test coupons were heated in an open vessel to a temperature of either 140° C. or 165° C. and any materials not condensed by the attached water-cooled reflux condenser were allowed to escape during the course of the test. The results are shown in the table below.

TABLE 3
Corrosion Tests - Conditions: 168 hrs in Glycerol Hydrochlorination
Reaction Product (DCH, HCl, Water).

Temperature

| Metal Coupon | 140° C. | 165° C. |
|---|---|---|
| Hastelloy C3 | 2.15 | 0.31 |
| Hastelloy B4 | 0.45 | 0.34 |

Values in Table Are % Mass Lost

1. A process for converting at least one multihydroxylated-aliphatic hydrocarbon and/or an ester thereof to at least one chlorohydrin and/or an ester thereof, comprising at least one reaction step in which the multihydroxylated-aliphatic hydrocarbon and/or ester thereof is contacted with hydrogen chloride under reaction conditions to produce the chlorohydrin and/or ester thereof; wherein the reaction step provides at least a first effluent of the reaction step; wherein the concentration of hydrogen chloride in the at least first effluent of the reaction step is below 0.8% by weight; wherein the reaction step is followed by at least one downstream processing step in which the at least first effluent of the reaction step is processed; and wherein the downstream processing step is performed under conditions such that the at least first effluent of the reaction step containing the chlorohydrin and/or ester thereof is kept at a temperature of less than 120° C.

2. The process of claim 1, wherein the downstream processing equipment used in said downstream processing step is made of or covered with corrosion resistant material in the only areas where such downstream processing equipment is in contact with an effluent whose total hydrogen chloride concentration is greater than 0.8% by weight, relative to the total weight of said effluent.

3. The process of claim 1, wherein in the downstream processing step, the water is removed from the effluents of the reaction step.

4. The process of claim 3, wherein the water is removed by a reactive, cryogenic, extractive, azeotropic, absorptive or evaporative in-situ or ex-situ technique.

5. The process of claim 4, wherein the concentration of hydrogen chloride in the effluents of the reaction step is reduced by dilution, neutralization, stripping, extraction, absorption, or distillation.

6. The process of claim 1, wherein the total fluoride concentration in each process stream or feed stream is limited to less than 50 ppm by weight;
and wherein the fluoride concentration is reduced by a treatment using a fluoride scavenging agent, of a heterogeneous or of a homogenous nature.

7. The process of claim 1, wherein the reaction step is performed with superatmospheric partial pressure of hydrogen chloride; wherein the reaction step is performed with the absence of water removal; and wherein the hydrogen chloride source is hydrogen chloride gas.

8. The process of claim 1, wherein the chlorohydrin is a dichlorohydrin; and wherein the chlorohydrin is 1,3-dichloro-propan-2-ol, or 2,3-dichloropropan-1-ol, or a mixture thereof.

9. The process of claim 1, wherein the multihydroxylated-aliphatic hydrocarbon comprises at least one compound chosen from 1,2-ethanediol; 1,2-propanediol; 1,3-propanediol; 1-chloro-2,3-propanediol; 2-chloro-1,3-propanediol; 1,4-butanediol; 1,5-pentanediol; cyclohexanediols; 1,2-butanediol; 1,2-cyclohexanedimethanol; 1,2,3-propanetriol; and mixtures thereof.

10. The process of claim 1, wherein a catalyst is used in the reaction step; wherein the catalyst is chosen from a carboxylic acid; an anhydride; an acid chloride; an ester; a lactone; a lactam; an amide; a metal organic compound; or a combination thereof; or wherein the catalyst is an acid with a functional group consisting of a halogen, an amine, an alcohol, an alkylated amine, a sulfhydryl, an aryl group or an alkyl group, or combinations thereof, wherein this moiety is not sterically hindering the carboxylic acid group.

11. The process of claim 10, wherein the catalyst is a carboxylic acid, an ester of a carboxylic acid, or a combination thereof; wherein the catalyst is acetic acid; or wherein the catalyst is chosen from caprolactone, 6-hydroxyhexanoic acid, 6-chlorohexanoic, an ester thereof, or a mixture thereof.

12. The process of claim 2, wherein the corrosion resistant material is chosen from alloys containing at least one metal chosen from tantalum, zirconium, platinum, titanium, gold, silver, nickel, niobium, molybdenum and mixtures thereof; wherein the corrosion resistant material is chosen from ceramics or metallic-ceramics, refractory materials, graphite, glass-lined materials; wherein the corrosion resistant material is a polymer chosen from polyolefins, fluorinated polymers, polymers containing sulfur and/or aromatics, epoxy resins, phenolic resins, vinyl ester resins, furan resins or mixtures thereof; or wherein the corrosion resistant material is chosen from enameled steels.

13. The process of claim 2, wherein the corrosion resistant material is used to make the actual body of the downstream processing equipment devices which need to be protected from corrosion; or wherein the corrosion resistant material is used as a coating of the surface of the downstream processing equipment devices which need to be protected from corrosion.

14. The process of claim 7, wherein the reaction step is carried out at a partial pressure of hydrogen chloride of from 103 kPa to 6900 kPa; and wherein the reaction step is carried out at a temperature of from 25° C. to 300° C.

15. The process of claim 1, wherein the equipment used to perform the reaction step is at least partially made of or covered with corrosion resistant material; or wherein the equipment used to perform the reaction step is totally made of or covered with corrosion resistant material.

16. A process for reducing corrosion in equipment located downstream of a hydrochlorination reaction zone in which at least one multihydroxylated-aliphatic hydrocarbon and/or an ester thereof is converted into at least one chlorohydrin and/or an ester thereof; wherein the reaction zone provides at least a first effluent of the reaction zone; wherein the concentration of hydrogen chloride in the at least first effluent of the reaction zone is below 0.8% by weight; and wherein the at least first effluent of the reaction zone containing the chlorohydrin and/or ester thereof is kept at a temperature of less than 120° C.

17. The process of claim 16, wherein the water is removed from the effluent of the reaction zone.

18. An installation for converting at least one multihydroxylated-aliphatic hydrocarbon and/or an ester thereof to at least one chlorohydrin and/or an ester thereof, comprising at least one reaction unit in which the multihydroxylated-aliphatic hydrocarbon and/or ester thereof is contacted with hydrogen chloride under reaction conditions to produce the chlorohydrin and/or ester thereof; said reaction unit being connected to at least one downstream processing unit in which the effluent of the reaction unit is processed and/or stored; wherein the equipment used in said downstream processing unit is made of or covered with non-corrosion resistant material in the only areas where such equipment is in contact with an effluent whose total hydrogen chloride concentration is less than 0.8% by weight, relative to the total weight of said effluent; and wherein the equipment used in said downstream processing unit is made of or covered with corrosion resistant material in the only areas where such equipment is in contact with an effluent whose total hydrogen chloride concentration is greater than 0.8% by weight, relative to the total weight of said effluent.
